(19) United States
(12) Patent Application Publication
(10) Pub. No.: US 2024/0398297 A1
(43) Pub. Date: Dec. 5, 2024

(54) BLOOD COLLECTION SYSTEM AND BLOOD COLLECTION METHOD

(71) Applicant: Hitachi, Ltd., Tokyo (JP)

(72) Inventors: Masahiro KATO, Tokyo (JP); Akio NAGASAKA, Tokyo (JP); Naoto MIURA, Tokyo (JP); Yo NONOMURA, Tokyo (JP); Yusuke MATSUDA, Tokyo (JP); Keiichiro NAKAZAKI, Tokyo (JP)

(21) Appl. No.: 18/689,039

(22) PCT Filed: Aug. 26, 2022
(86) PCT No.: PCT/JP2022/032181

§ 371 Date: Mar. 4, 2024

(30) Foreign Application Priority Data
Sep. 29, 2021 (JP) 2021-158605

(51) Int. Cl. A61B 5/15; A61B 5/1171; G06V 40/145

(52) U.S. Cl. CPC A61B 5/150068; A61B 5/1171; A61B 5/15003; G06V 40/145

(57) ABSTRACT

In order to solve such a problem, the invention provides a blood collection system used for collecting blood of a subject and a blood collection method executed in the blood collection system. The blood collection system punctures a finger of the subject with a puncture needle; and then supplies pressurized air to a cuff attached to the finger of the subject in a rolling manner so as to inflate the cuff and apply a predetermined pressure to the finger of the subject.

### TECHNICAL FIELD

The present invention relates to a blood collection system and method, and is suitably applied to an authentication and blood collection system that simultaneously authenticates a subject and collects blood.

### BACKGROUND ART

In recent years, an increase in lifestyle-related diseases and predispositions thereof has become a social problem. Along with this, there are increasing opportunities to collect blood to diagnose presence or absence of lifestyle-related diseases such as diabetes, hyperlipidemia, and liver dysfunction. For example, a blood test is performed in most cases during a complete medical checkup or a health examination.

In recent years, comprehensive diagnosis services for lifestyle-related diseases have been provided by private companies that set up booths in shopping malls, sports clubs, entertainment facilities, corporate offices, or the like and perform health checks including blood tests at a low price (see NPL 1).

Here, as a technique related to a blood test, PTL 1 discloses a technique of performing subject authentication, blood collection, and measurement using a vein pattern of a hand by a test device equipped with a camera.

### CITATION LIST

### Patent Literature

PTL 1: JP2020-510812A

### Non Patent Literature

NPL 1: “Preventive Medical Business”, [online], Carepro Co., Ltd., [searched on Sep. 14, 2021], Internet <https://carepro.co.jp/preventive/>

### SUMMARY OF INVENTION

### Technical Problem

For example, in NPL 1, a subject collects blood by himself or herself when conducting a blood collection test, and blood collection performed by the subject who is not a specialist such as a doctor or a nurse may not be able to collect a sufficient amount of blood.

The invention has been made in view of the above points, and an object thereof is to provide a blood collection system and method capable of collecting a sufficient amount of blood by improving blood collection efficiency.

### Solution to Problem

In order to solve such a problem, the invention provides a blood collection system used for collecting blood of a subject, and the blood collection system includes: a puncturing and blood collection mechanism configured to puncture a finger of the subject with a puncture needle; a cuff configured to be attached to the finger of the subject in a rolling manner; and a cuff control unit configured to supply pressurized air to the cuff so as to inflate the cuff and apply a predetermined pressure to the finger of the subject.

The invention provides a blood collection method executed in a blood collection system used for collecting blood of a subject, and the blood collection method includes: a first step in which the blood collection system punctures a finger of the subject with a puncture needle; and a second step in which the blood collection system supplies pressurized air to a cuff attached to the finger of the subject in a rolling manner so as to inflate the cuff and apply a predetermined pressure to the finger of the subject.

### Advantageous Effects of Invention

According to the invention, it is possible to implement a blood collection system and method capable of collecting a sufficient amount of blood by improving blood collection efficiency.

### BRIEF DESCRIPTION OF DRAWINGS

FIG. 1 is a block diagram showing an overall configuration of a blood collection system according to the present embodiment.

FIG. 2(A) and (B) of FIG. 2 are a top view and a side view showing a puncturing and blood collection mechanism of a blood collection device.

FIG. 3 is a flowchart showing processing procedures of authentication and blood collection control processing.

FIG. 4 is a flowchart showing processing procedures of brightness adjustment processing.

FIG. 5 is a diagram showing an image example of a fingertip image.

FIG. 6 is a graph showing a relationship between an average luminance in a processing region and a light adjustment amount.

FIG. 7 is a flowchart showing processing procedures of subject authentication processing.

FIG. 8(A) of FIG. 8 is a diagram showing a puncture needle trajectory, and (B) of FIG. 8 is a chart showing puncture needle trajectory coordinate data.

FIG. 9 is a diagram showing a method for obtaining a puncture needle trajectory.

FIG. 10 is a flowchart showing processing procedures of puncture position determination processing.

FIG. 11(A) of FIG. 11 is a diagram and a graph showing a method for extracting a puncture position candidate, and (B) of FIG. 11 is a diagram showing a method for detecting a valley portion of a graph.

FIG. 12 is a diagram showing a configuration of a cuff control unit.

FIG. 13(A) of FIG. 13 is a graph showing a waveform of a control voltage applied to a pump of the cuff control unit, (B) of FIG. 13 is a graph showing changes in a cuff pressure, (C) of FIG. 13 is a graph showing a waveform of a control voltage applied to an infrared LED, and (D) of FIG. 13 is a graph showing brightness of near-infrared light emitted from the infrared LED.

FIG. 14(A) and (B) of FIG. 14 are diagrams showing a vein enhancement effect attained by pressing a finger of a subject.

### DESCRIPTION OF EMBODIMENTS

Hereinafter, an embodiment of the invention will be described in detail with reference to the drawings.

### (1) Configuration of Blood Collection System According to Present Embodiment

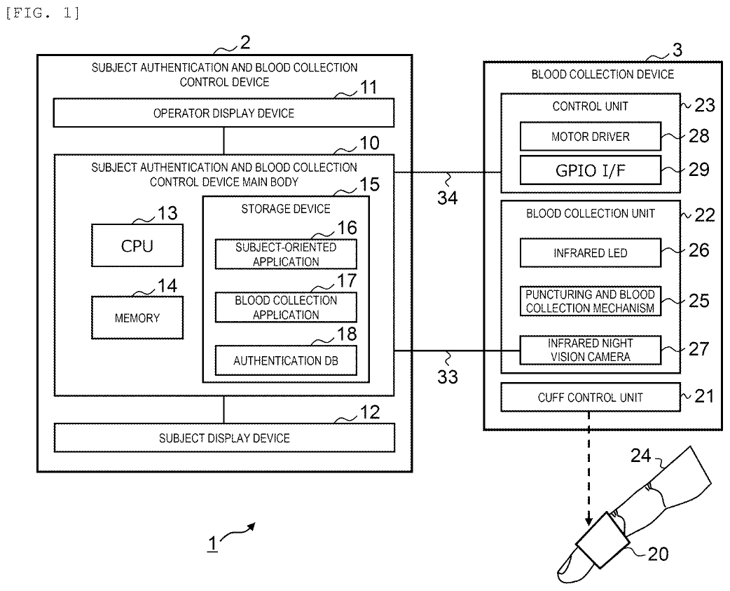

In FIG. 1, reference numeral 1 denotes a blood collection system according to the present embodiment as a whole. The blood collection system 1 is a blood collection system that simultaneously authenticates a subject and collects blood, and includes a subject authentication and blood collection control device 2 and a blood collection device 3.

The subject authentication and blood collection control device 2 includes a subject authentication and blood collection control device main body 10, an operator display device 11, and a subject display device 12. The subject authentication and blood collection control device main body 10 is implemented by a general-purpose computer including a central processing unit (CPU) 13, a memory 14, and a storage device 15.

The CPU 13 is a processor that controls an operation of the entire subject authentication and blood collection control device 2. The memory 14 is implemented by, for example, a volatile semiconductor memory, and is used as a work memory of the CPU 13. Further, the storage device 15 is implemented by a nonvolatile large-capacity storage device such as a hard disk device or a solid state drive (SSD), and stores, in advance, various types of software and various types of data that require long-term storage.

The various types of software stored in the storage device 15 are loaded into the memory 14 when the subject authentication and blood collection control device 2 is started or when necessary, and the CPU 13 executes programs loaded into the memory 14, whereby various types of processing are executed in the entire subject authentication and blood collection control device 2 as will be described later.

The operator display device 11 is a display device used to display information for an operator who operates the subject authentication and blood collection control device 2, and includes a liquid crystal panel, an organic electro-luminescence (EL), a touch panel, or the like. The subject display device 12 is a display device used for displaying information for the subject, and includes, for example, a touch panel.

The storage device 15 of the subject authentication and blood collection control device main body 10 stores subject-oriented application software (hereinafter referred to as a subject-oriented application) 16, blood collection application software (hereinafter referred to as a blood collection application) 17, and an authentication database 18.

The subject-oriented application 16 is software capable of communicating information with the blood collection application 17, and displays a sign of blood collection start or a sign of blood collection completion on the subject display device 12 according to an instruction from the blood collection application 17, and displays various types of information such as a usage guide on the subject display device 12 according to an operation input by the subject to the subject display device 12.

The blood collection application 17 executes processing of adjusting brightness of near-infrared light emitted from an infrared light emitting diode (LED) 26 of the blood collection device 3, which will be described later, authenticating the subject, and determining a position on a finger of the subject punctured with a puncture needle (hereinafter referred to as a puncture position).

The authentication database 18 is a database in which feature images of finger veins of pre-registered subjects are stored in association with identifiers of the corresponding subjects (hereinafter referred to as “subject IDs”). Details of the “feature images of the finger veins” will be described later.

The blood collection device 3 is a device that collects blood from the subject, and includes a cuff 20, a cuff control unit 21, a blood collection unit 22, and a control unit 23.

The cuff 20 is a balloon body formed in a band shape, and is used by being attached to a finger 24 of the subject in a rolling manner. The cuff control unit 21 is a driving device of the cuff 20 that increases an internal pressure of the cuff 20 by injecting pressurized air into the cuff 20 so as to inflate the cuff 20, or reduces the internal pressure of the cuff 20 by stopping supply of the pressurized air to the cuff 20 so as to deflate the cuff 20. By inflating or deflating the cuff 20, it is possible to apply a pressure to the finger 24 rolled with the cuff 20 or release the pressure.

The blood collection unit 22 is a mechanism portion that collects blood by puncturing the finger 24 of the subject with a puncture needle, and includes a puncturing and blood collection mechanism 25, the infrared LED 26, and an infrared night vision camera 27.

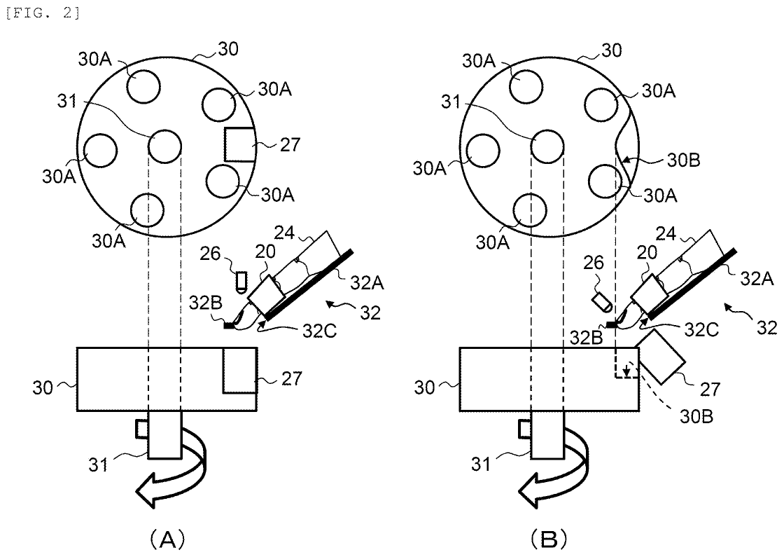

As shown in (A) of FIG. 2, the puncturing and blood collection mechanism 25 includes a rotary table 30 provided with a plurality of holders 30A each formed of a cylindrical recess. Each holder 30A is formed at an equal distance from a central position of the rotary table 30.

A shaft body 31 is fixed to a central portion of the rotary table 30 coaxially with the rotary table 30. The shaft body 31 is engaged with an actuator such as a motor (not shown), and by driving the actuator, the shaft body 31 and the rotary table 30 can be integrally driven to rotate around a central axis of the shaft body 31 and the rotary table 30.

A finger rest 32 is disposed above the rotary table 30. As shown in FIG. 2, the finger rest 32 includes a sloped portion 32A that slopes downward toward a tip end thereof, and a fingertip support portion 32B provided continuously with the sloped portion 32A on a tip end side of the sloped portion 32A. The finger rest 32 has an opening 32C at a position facing a finger pad portion on a tip end side of a first joint of the finger 24 placed on the finger rest 32 in a predetermined state.

Accordingly, when the subject places the finger 24 on the finger rest 32 in the predetermined state in which a tip end portion of the finger 24 abuts against a tip end surface of the opening 32C as shown in (A) of FIG. 2, the finger pad portion of the finger 24 on the tip end side of the first joint is exposed to a rotary table 30 side through the opening 32C.

The infrared LED 26 is a light source that irradiates the finger 24 of the subject with near-infrared light having a wavelength of about 940 nm. As shown in (A) of FIG. 2, the infrared LED 26 is disposed above the finger rest 32 such that the emitted near-infrared light passes obliquely (obliquely with respect to the finger 24) through a portion beyond the first joint of the finger 24 of the subject placed on the finger rest 32 as described above and then is emitted downward through the opening 32C of the finger rest 32.

The infrared night vision camera 27 is fixed to the rotary table 30 of the puncturing and blood collection mechanism 25 such that an imaging optical axis thereof overlaps an optical axis of the infrared LED 26 in order to image the near-infrared light that passes through the finger 24 of the subject and is emitted downward through the opening 32C of the finger rest 32 as described above. The infrared night vision camera 27 is connected to the subject authentication and blood collection control device main body 10 via a universal serial bus (USB) cable 33 (FIG. 1), video data on a captured video obtained by such imaging is transmitted to the subject authentication and blood collection control device main body 10 in real time via the USB cable 33.

The control unit 23 is a computer including information processing resources such as a CPU and a memory (not shown). The control unit 23 is connected to the subject authentication and blood collection control device main body 10 via a local area network (LAN) cable 34 (FIG. 1), and controls the puncturing and blood collection mechanism 25 according to an instruction given from the subject authentication and blood collection control device main body 10 via the LAN cable 34.

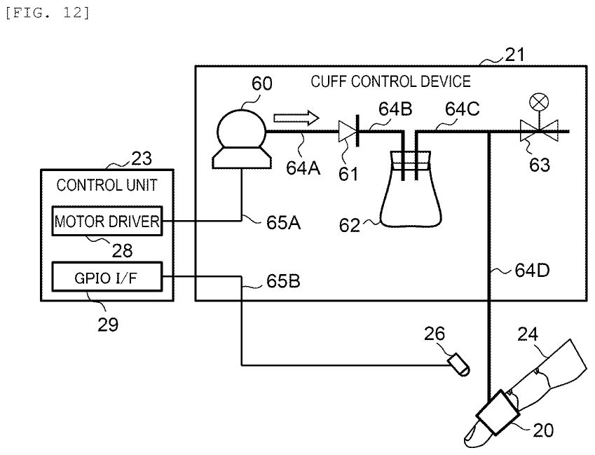

The control unit 23 includes a motor driver 28 and a general purpose input/output (GPIO) interface 29. Then, the control unit 23 drives and controls a pump 60 of the cuff control unit 21, which will be described later with reference to FIG. 12, by the motor driver 28 and controls brightness of the infrared LED 26 connected to the GPIO interface 29 according to an instruction given from the subject authentication and blood collection control device main body 10.

(B) of FIG. 2 shows another configuration example of the blood collection unit 22, in which portions corresponding to those in (A) of FIG. 2 are denoted by the same reference signs. This configuration example is different from (A) of FIG. 2 in that the infrared LED 26 emits light perpendicularly (perpendicularly to the finger 24) through the portion beyond the first joint of the finger 24 of the subject placed on the finger rest 32.

Accordingly, the infrared night vision camera 27 is disposed separately from the rotary table 30 such that an imaging optical axis thereof overlaps an optical axis of the infrared LED 26. Therefore, the rotary table 30 has a recess 30B for avoiding interference with the infrared night vision camera 27 during rotation.

In a case of the present embodiment, both (A) of FIG. 2 and (B) of FIG. 2 can be adopted as a configuration of the blood collection unit 22, but since a contrast of an image tends to be lower in a configuration in (A) of FIG. 2, a configuration in (B) of FIG. 2 is generally adopted in many cases. However, in the present embodiment, the configuration in (A) of FIG. 2 in which a mechanism of the blood collection unit 22 can be made more simplified and compact than the configuration in (B) of FIG. 2 is given priority.

### (2) Processing by Subject Authentication and Blood Collection Control Device Related to Subject Authentication and Blood Collection

Next, a flow of a series of processing (hereinafter referred to as subject authentication and blood collection processing) executed in the subject authentication and blood collection control device 2 that authenticates the subject and collects blood in the blood collection system 1 will be described. In the following description, a processing body of the subject authentication and blood collection processing will be described as the blood collection application 17 (FIG. 1), but in practice, the CPU 13 (FIG. 1) of the subject authentication and blood collection control device main body 10 (FIG. 1) executes the processing based on the blood collection application 17.

### (2-1) Subject Authentication and Blood Collection Control Processing

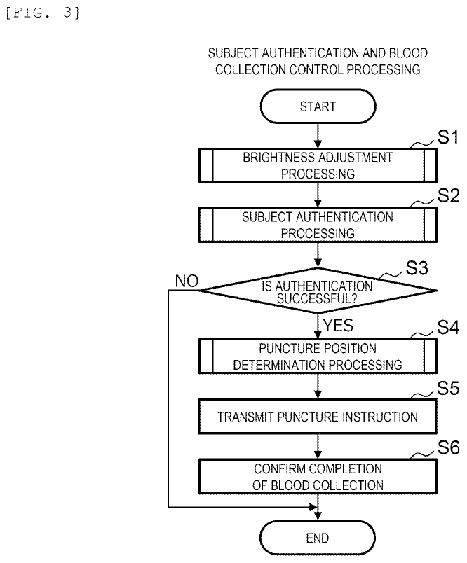

FIG. 3 shows a flow of a series of the subject authentication and blood collection control processing. When a predetermined operation is performed by the operator of the subject authentication and blood collection control device 2 after the subject places the finger 24 on the finger rest 32 ((A) of FIG. 2) in a predetermined state, the blood collection application 17 starts the subject authentication and blood collection control processing. Then, the blood collection application 17 first adjusts brightness of the near-infrared light emitted from the infrared LED 26 (FIG. 1) of the blood collection device 3 (FIG. 1) based on video data on a captured video transmitted from the infrared night vision camera 27 (FIG. 1) of the blood collection device 3 (S1).

Subsequently, the blood collection application 17 authenticates the subject based on the video data (S2). When the authentication fails, the blood collection application 17 ends the subject authentication and blood collection control processing (S3; NO).

On the other hand, when subject authentication is successful in step S2 (S3; YES), the blood collection application 17 determines a position (hereinafter referred to as a puncture position), on a finger pad portion on a tip end side of the finger 24 of the subject exposed to the rotary table 30 side through the opening 32C ((A) of FIG. 2) of the finger rest 32, to be punctured with a puncture needle (S4).

Next, the blood collection application 17 transmits coordinate data on the puncture position determined in step S4 (hereinafter referred to as puncture position coordinate data) to the control unit 23 (FIG. 1) of the blood collection device 3 together with a puncture instruction to puncture the puncture position with the puncture needle (S5).

Thus, the control unit 23 of the blood collection device 3, which receives the puncture instruction, controls the puncturing and blood collection mechanism 25 based on the puncture position coordinate data given at this time, thereby rotating the rotary table 30 as necessary such that the puncture needle of a lancet (not shown), which is set in a manner of being fitted into one of the holders 30A ((A) of FIG. 2) of the rotary table 30, is located directly below the designated puncture position.

Thereafter, the control unit 23 raises the rotary table 30 such that the puncture position on the finger pad portion on the tip end side of the finger 24 of the subject placed on the finger rest 32 is punctured with the puncture needle of the lancet about 2 mm in depth. Accordingly, the puncture position determined by the blood collection application 17 is punctured with the puncture needle of the lancet, whereby blood is collected.

Thereafter, when puncturing is completed, the control unit 23 of the blood collection device 3 notifies the subject authentication and blood collection control device 2 of completion of the puncturing (S6). Then, the blood collection application 17 of the subject authentication and blood collection control device 2 that receives the notification ends the series of the subject authentication and blood collection control processing.

### (2-2) Brightness Adjustment Processing

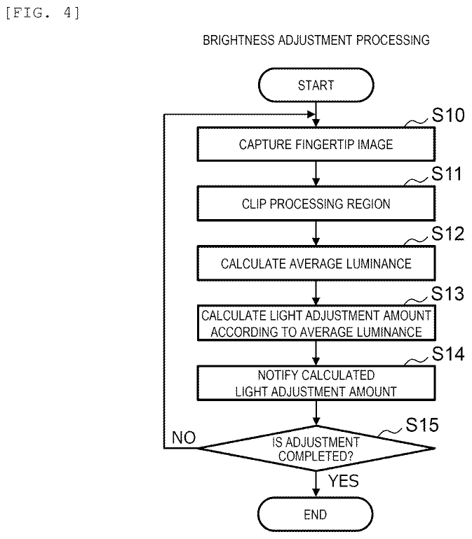

FIG. 4 shows specific processing contents of brightness adjustment processing executed by the blood collection application 17 in step S1 of the subject authentication and blood collection control processing described above with reference to FIG. 3. The blood collection application 17 adjusts the brightness of the near-infrared light emitted from the infrared LED 26 of the blood collection device 3 according to the processing procedures shown in FIG. 4.

In practice, the blood collection application 17 starts the brightness adjustment processing when proceeding to step S2 of the subject authentication and blood collection control processing, and first captures an image of a fingertip of the subject (hereinafter referred to as a fingertip image) based on the video data on the captured video transmitted from the infrared night vision camera 27 (S10).

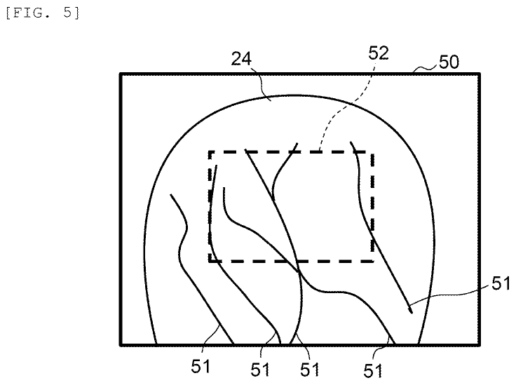

FIG. 5 shows an image example of a fingertip image 50 captured at this time. The tip end portion of the finger 24 of the subject exposed to an infrared night vision camera 27 side through the opening 32C ((A) of FIG. 2) of the finger rest 32 ((A) of FIG. 2) and shadows of finger veins 51 present in the tip end portion are projected in the fingertip image 50.

In this case, in the present embodiment, since the infrared LED 26 that emits the near-infrared light having a wavelength of about 940 nm is used, only the shadows of the finger veins 51 present at the same position as a depth of about 2 mm from a surface of the finger pad portion at the tip end portion of the finger 24 of the subject, that is, a depth at which the finger 24 of the subject is punctured with the puncture needle of the lancet in the present embodiment, are projected in the fingertip image 50.

Subsequently, the blood collection application 17 clips a preset region corresponding to the vicinity of the tip end portion of the finger 24 of the subject in the captured fingertip image 50 from the fingertip image 50 as a processing region 52 for the processing after step S12 (S11), and calculates an average luminance in the clipped processing region 52 (S12).

Next, the blood collection application 17 calculates a light adjustment amount according to the calculated average luminance in the processing region 52 (S13). A method for calculating the light adjustment amount at this time will be described later. Then, the blood collection application 17 transmits an instruction to adjust the brightness of the near-infrared light emitted from the infrared LED 26 (hereinafter referred to as a light adjustment instruction) to the control unit 23 of the blood collection device 3 together with the light adjustment amount at this time calculated in step S13 (S14). Thus, the control unit 23 of the blood collection device 3 that receives the notification controls the infrared LED 26 to change the brightness by the notified light adjustment amount.

Thereafter, the blood collection application 17 determines whether brightness adjustment for the near-infrared light emitted from the infrared LED 26 is completed (S15). Specifically, similarly to steps S10 to S13, the blood collection application 17 newly captures the fingertip image 50, calculates an average luminance in the processing region 52 clipped from the captured fingertip image 50, and determines whether the calculated average luminance falls within a predetermined range based on a preset target luminance.

When obtaining a negative result in this determination, the blood collection application 17 returns the processing to step S10, and thereafter repeats the processing in steps S10 to S15 until obtaining a positive result in step S15. When the positive result is obtained in step S15 because the average luminance in the processing region 52 of the fingertip image 50 eventually falls within the predetermined range based on the target luminance, the blood collection application 17 ends the brightness adjustment processing.

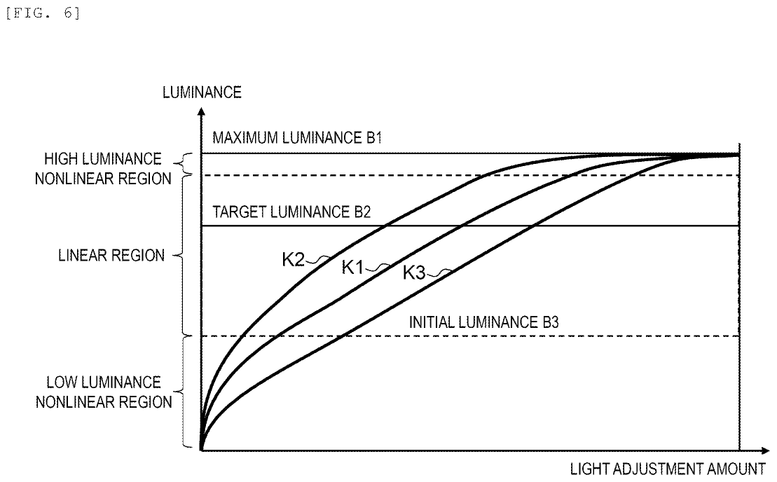

Here, specific processing contents by the blood collection application 17 in step S13 of the brightness adjustment processing will be described. FIG. 6 shows a relationship between an average luminance in the processing region 52 preset in the blood collection application 17 and a light adjustment amount of the near-infrared light emitted from the infrared LED 26 with respect to the average luminance, in the present embodiment.

In the present embodiment, the average luminance in the processing region 52 is divided into three regions: a high luminance nonlinear region, a linear region, and a low luminance nonlinear region, and different methods for calculating the light adjustment amount are applied to the high luminance nonlinear region, the linear region, and the low luminance nonlinear region. However, this region classification is a guideline assuming a standard case.

In FIG. 6, a curve K1 indicates a relationship between an average luminance in the processing region 52 assuming a finger smaller than a standard and a light adjustment amount of the infrared LED 26 with respect to the average luminance, a curve K2 indicates a relationship between an average luminance in the processing region 52 assuming a finger having a standard size (hereinafter referred to as a standard finger) and a light adjustment amount of the infrared LED 26 with respect to the average luminance, and a curve K3 indicates a relationship between an average luminance in the processing region 52 assuming a finger larger than the standard and a light adjustment amount of the infrared LED 26 with respect to the average luminance. Here, it is assumed that slopes of the curves K1 to K3 are substantially equal regardless of the size of the finger.

When the average luminance calculated in step S12 of the brightness adjustment processing (FIG. 4) is within the high luminance nonlinear region and exceeds a preset maximum luminance B1 (FIG. 6), the blood collection application 17 calculates the light adjustment amount as ½ of the average luminance at that time.

When the average luminance calculated in step S12 is within the low luminance nonlinear region, the blood collection application 17 proportionally divides a maximum light adjustment amount (hereinafter referred to as a maximum light adjustment amount), which can be adjusted based on a ratio of an initial luminance B3 (FIG. 3) that is a boundary between the linear region and the low luminance nonlinear region to the above-described maximum luminance B1, and sets the one corresponding to the maximum luminance B1, in the maximum light adjustment amount after the proportional division, as the light adjustment amount.

Further, when the average luminance is within the linear region, the blood collection application 17 increases the light adjustment amount in a stepwise manner until the average luminance calculated in step S12 reaches the preset target luminance B2 such that the brightness of the near-infrared light emitted from the infrared LED 26 gradually approaches the target luminance B2 (FIG. 6). Specifically, the blood collection application 17 calculates an average luminance Δy in the processing region 52 by the following equation 1 with a slope in the linear region of the curves K1 to K3 in FIG. 6 as a,

$$Δ y = \frac{TARGET LUMINANCE - AVERAGE LUMINANCE}{5} \qquad ( Equation 1 )$$

and uses the calculated Δy to calculate a current light adjustment amount Δx by the following equation 2.

$$Δ x = \frac{Δ y}{a} \qquad ( Equation 2 )$$

### (2-3) Subject Authentication Processing

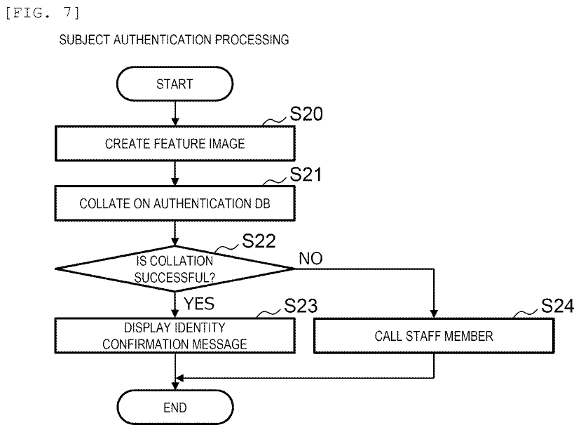

FIG. 7 shows specific processing contents of subject authentication processing executed by the blood collection application 17 in step S2 of the subject authentication and blood collection control processing described above with reference to FIG. 3. The blood collection application 17 authenticates the subject who places the finger 24 on the finger rest 32 at that time according to processing procedures shown in FIG. 7.

In practice, the blood collection application 17 starts the subject authentication processing when proceeding to step S2 of the subject authentication and blood collection control processing, and first creates a feature image in which a feature of finger veins of the subject is extracted from a frame image (that is, the fingertip image 50 (FIG. 5)) based on the video data transmitted from the infrared night vision camera 27 (S20).

Specifically, the blood collection application 17 sequentially executes inter-frame averaging processing of averaging a luminance of pixels at the same position in a plurality of frames with respect to the fingertip image 50 of the subject, Gaussian filtering processing of removing noise from the fingertip image 50, and image reduction processing of reducing the fingertip image 50, and creates the feature image by extracting a feature amount from the reduced image of the fingertip image 50 thus obtained.

Next, the blood collection application 17 collates the created feature image with a feature image of each subject stored in the authentication database 18 (FIG. 1) (S21), and determines whether the feature image created in step S20 matches any feature image registered in the authentication database 18 (S22).

Then, when obtaining a positive result in this determination, the blood collection application 17 instructs the subject-oriented application 16 (FIG. 1) to display, on the subject display device 12 (FIG. 1), a message for checking whether the current subject is a subject corresponding to the feature image on the authentication database 18, which matches the feature image created in step S20 in the collation in step S21 (S23), and thereafter ends the subject authentication processing.

Thus, in this case, the subject-oriented application 16 displays a message such as “It's Mr. XX” on the subject display device 12. “XX” is a name of the subject corresponding to the feature image on the authentication database 18, which matches the feature image created in step S20 in the collation in step S21.

On the other hand, when obtaining a negative result in the determination in step S22, the blood collection application 17 calls a staff member (S24), and thereafter ends the subject authentication processing.

### (2-4) Puncture Position Determination Processing

Next, puncture position determination processing executed by the blood collection application 17 in step S4 of the subject authentication and blood collection control processing described above with reference to FIG. 3 will be described. First, a puncture needle trajectory will be described.

As described above, in the blood collection system 1 according to the present embodiment, the lancet (not shown) is set by being inserted into the holder 30A of the rotary table 30 described above with reference to (A) of FIG. 2, and then the rotary table 30 is raised to puncture the finger pad portion of the fingertip of the subject exposed through the opening 32C ((A) of FIG. 2) of the finger rest 32 ((A) of FIG. 2) with the puncture needle of the lancet.

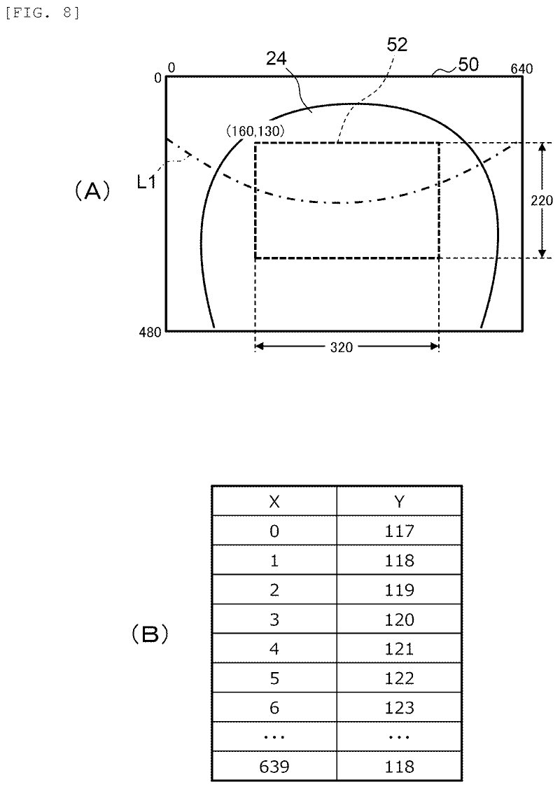

Therefore, since the puncture needle moves on circumference around the central axis of the rotary table 30 by rotating the rotary table 30, the puncture needle in the fingertip image 50 (FIG. 5) when the rotary table 30 is rotated moves while drawing an arc-shaped trajectory (hereinafter referred to as a puncture needle trajectory) L1 as shown in (A) of FIG. 8. Accordingly, the puncture needle trajectory L1 can be referred to as a collection of positions that can be punctured with the puncture needle of the lancet set in the holder 30A of the rotary table 30.

In this case, when a position of the infrared night vision camera 27 with respect to the rotary table 30 is fixed, the puncture needle trajectory L1 is normally at the same position in the fingertip image 50. Therefore, the puncture needle trajectory L1 in the fingertip image 50 can be obtained by the following method.

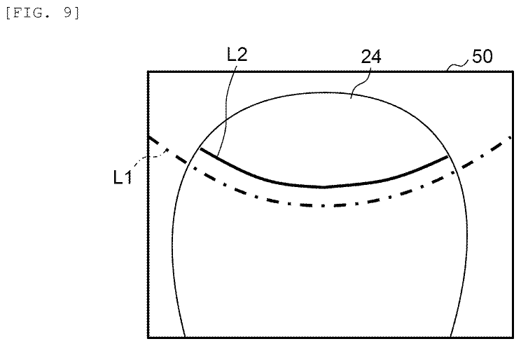

First, a felt-tip pen is set in a fixed state in the holder 30A of the rotary table 30 such that a pen tip is located at the same position as the puncture needle of the lancet set in the holder 30A of the rotary table 30. Next, the finger 24 is placed on the finger rest 32 in the predetermined state, and the rotary table 30 is rotated with the pen tip of the felt-tip pen in contact with the finger pad portion of the finger 24 exposed through the opening 32C of the finger rest 32. In this way, an arc-shaped marker (hereinafter referred to as a trajectory marker) L2 is drawn on the finger pad portion of the finger 24 as shown in FIG. 9.

In FIG. 9, the trajectory marker L2 and the puncture needle trajectory L1 are shown separated for ease of understanding, but in practice, the trajectory marker L2 is a part of the puncture needle trajectory L1, and thus the trajectory marker L2 completely overlaps the puncture needle trajectory L1. Accordingly, the puncture needle trajectory L1 in the fingertip image 50 can be obtained based on the trajectory marker L2.

In the case of the present embodiment, the puncture needle trajectory L1 obtained in this way is stored in advance by the control unit 23 of the blood collection device 3 as coordinate data (hereinafter collectively referred to as puncture needle trajectory coordinate data) on each point on the puncture needle trajectory L1 as shown in (B) of FIG. 8, for example. In the puncture needle trajectory coordinate data shown in (B) of FIG. 8, coordinates at positions on the puncture needle trajectory L1 (horizontal positions of 0th pixel, 1st pixel, 2nd pixel, . . . ) are obtained with an image size of the fingertip image 50 at 640×480 pixels and a size of the processing region at 320×220 pixels as shown in (A) of FIG. 8.

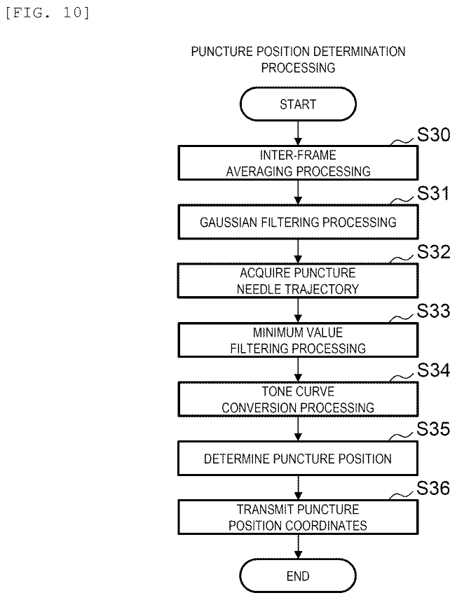

Here, FIG. 10 shows specific processing contents of the puncture position determination processing executed by the blood collection application 17 in step S4 of the subject authentication and blood collection control processing described above with reference to FIG. 3. The blood collection application 17 determines a puncture position of the finger 24 placed on the finger rest 32 according to processing procedures shown in FIG. 10.

In practice, the blood collection application 17 starts the puncture position determination processing when proceeding to step S4 of the subject authentication and blood collection control processing, and first sequentially executes the inter-frame averaging processing and the Gaussian filtering processing described above on the fingertip image based on the video data transmitted from the infrared night vision camera 27 of the blood collection device 3 (S30 and S31).

The blood collection application 17 accesses the control unit 23 of the blood collection device 3 to acquire the puncture needle trajectory coordinate data described above with reference to (B) of FIG. 8 (S32), and sequentially executes, on the acquired puncture needle trajectory coordinate data, minimum value filtering processing of converting into a pixel having the smallest value by comparing values of surrounding pixels, and tone curve conversion processing of converting a density of the image (S33 and S34).

Subsequently, the blood collection application 17 detects several positions, on the puncture needle trajectory L1 based on the puncture needle trajectory coordinate data subjected to the minimum value filtering processing and the tone curve conversion processing as described above, intersecting with shadows of the finger veins 51 (FIG. 5) of the subject in the processing region 52 of the fingertip image 50, where shadows of the thicker finger veins 51 intersect with the puncture needle trajectory L1, and determines one position among coordinates at the detected positions as the puncture position (S35).

By setting the position where the shadow of the finger vein 51 of the subject intersects with the puncture needle trajectory L1 as the puncture position in this way, the finger vein 51 of the subject can be punctured for blood collection accordingly, and thus more blood can be collected than when blood is collected by puncturing a position other than the finger vein 51.

Then, the blood collection application 17 notifies the control unit 23 of the blood collection device 3 of coordinates at the puncture position determined in step S35 (S36), and then ends the puncture position determination processing.

Thus, the control unit 23 of the blood collection device 3 that receives the notification controls the puncturing and blood collection mechanism 25 of the blood collection unit 22 to locate the puncture needle of the lancet set on the rotary table 30 at the position notified from the blood collection application 17, and collects blood by raising the rotary table 30 so as to puncture the position with the puncture needle.

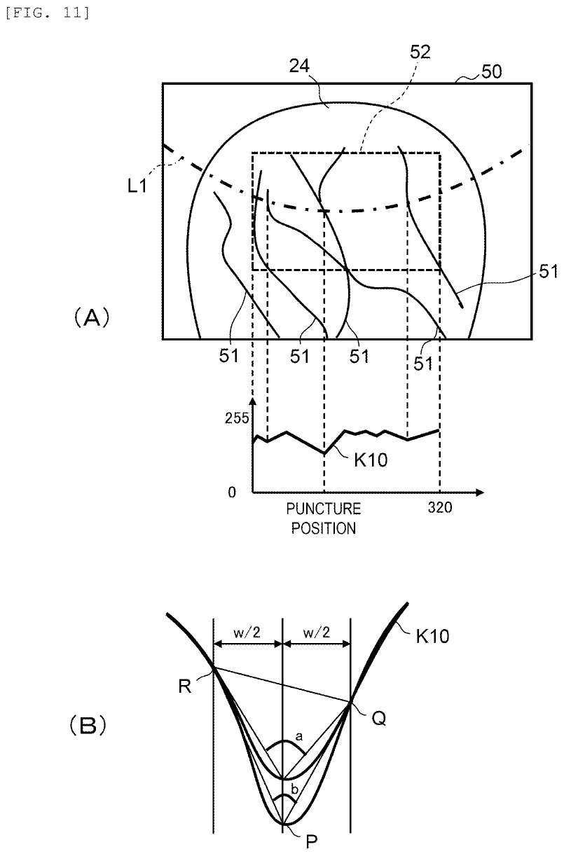

Specific processing contents by the blood collection application 17 in step S35 of the puncture position determination processing will be described. As shown in a graph K10 in a lower part of (A) of FIG. 11, a luminance of the puncture needle trajectory L1 in the processing region 52 is lower at a position intersecting with the shadow of the finger vein 51 than a surrounding luminance, and the thicker the intersecting finger vein 51, the lower the luminance.

As shown in (B) of FIG. 11, when a graph shape of the graph K10 in (A) of FIG. 11 is such that a position of the lowest point of a downwardly protruding portion (hereinafter referred to as a valley portion) is P, a point on the graph K10 corresponding to a position separated from the point P by a predetermined distance w/2 to a right side of the fingertip image 50 is Q, and a point on the graph K10 corresponding to a position separated from the point P by the predetermined distance w/2 to a left side of the fingertip image 50 is R, a magnitude of <QPR decreases as a depth of the corresponding valley portion increases (that is, as a luminance at a position corresponding to the lowest point in the fingertip image 50 decreases).

Therefore, the blood collection application 17 uses these principles to extract all valley portions whose luminance is smaller than that of a surrounding portion by a predetermined value or more from among portions on the puncture needle trajectory L1 passing through the processing region 52 of the fingertip image 50, and to extract several (for example, three) valley portions with smaller luminance (with a smaller angle QPR in (B) of FIG. 11) from among the extracted valley portions as puncture position candidates.

Then, the blood collection application 17 determines, as a current puncture position, at least a puncture position candidate different from the position punctured with the puncture needle during the previous blood collection, among the puncture position candidates extracted in this manner.

For this purpose, the blood collection application 17 stores the puncture position during the previous blood collection for each registered subject. Specifically, the blood collection application 17 performs management by registering, in the authentication database 18 (FIG. 1), a rank of a puncture position candidate with the lowest luminance determined as the puncture position, counting from the puncture position candidates with the lowest luminance during the previous blood collection (a luminance rank of the puncture position determined at that time among the puncture position candidates at that time) in association with the subject.

After extracting the puncture position candidates as described above, the blood collection application 17 refers to the authentication database 18, selects, by a round robin method or the like, one puncture position candidate having a luminance rank different from a luminance rank of the previous puncture position from among several puncture position candidates extracted this time, and determines the selected puncture position candidate as the current puncture position.

As described above, in the blood collection system 1 according to the present embodiment, at least a position different from the previous puncture position is selected as the puncture position for the puncture needle, thereby reducing pain experienced by the subject due to being punctured at the same position over and over again during blood collection.

### (3) Pressure Control Over Cuff and Brightness Control Over Infrared LED by Control Unit of Blood Collection Device

Next, pressure control over the cuff 20 (FIG. 1) and brightness control over the infrared LED executed by the control unit 23 of the blood collection device 3 will be described. In this case, first, a configuration of the cuff control unit 21 (FIG. 1) will be described.

FIG. 12 shows the configuration of the cuff control unit 21 of the blood collection device 3. As shown in FIG. 12, the cuff control unit 21 includes the pump 60, a check valve 61, an air buffer 62, and a pressure adjustment valve 63.

The pump 60 is an air circuit element that generates pressurized air, and the check valve 61 is an air circuit element that prevents a backflow of air due to pressure changes in an air circuit. The air buffer 62 is an air circuit element that reduces the pain of the subject due to rapid pressure changes in the cuff 20, and the pressure adjustment valve 63 is an air circuit element used for preventing the cuff 20 from rupturing by releasing an excessive pressure.

Further, the pump 60 and the check valve 61, the check valve 61 and the air buffer 62, and the air buffer 62 and the pressure adjustment valve 63 are connected by polypropylene (PP) tubes 64A, 64B, and 64C of φ 3 mm to 5 mm, respectively, and the air buffer 62 and the pressure adjustment valve 63 are branched and connected to the cuff 20 via a PP tube 64D of φ 3 mm to 5 mm.

In addition, the pump 60 is connected to the motor driver 28 of the control unit 23 via a jumper wire 65A. The pump 60 generates pressurized air based on a control voltage applied from the motor driver 28 via the jumper wire 65A, and outputs the generated pressurized air to the check valve 61 via the PP tube 64A. Then, the pressurized air is injected into the cuff 20 via the check valve 61 and the air buffer 62.

The infrared LED 26 is connected to the GPIO interface 29 of the control unit 23 via a jumper wire 65B. The infrared LED 26 is driven based on a control voltage applied from the GPIO interface 29 of the control unit 23 via the jumper wire 65B, and emits near-infrared light having brightness corresponding to the control voltage.

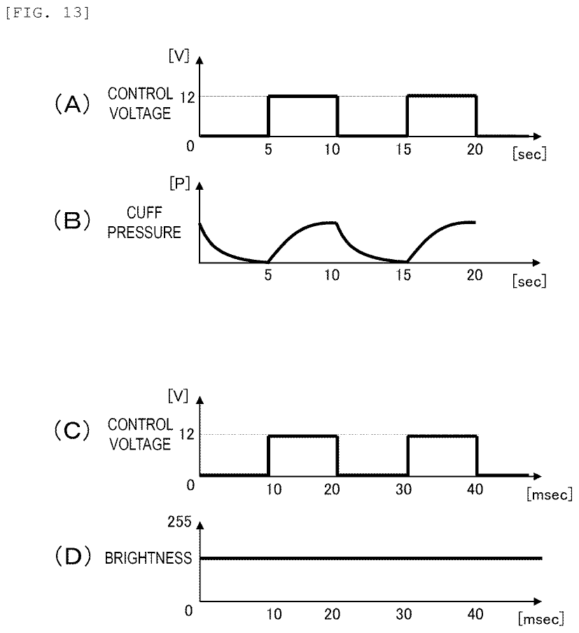

(A) of FIG. 13 shows a voltage waveform of a control voltage applied from the motor driver 28 of the control unit 23 to the pump 60 via the jumper wire 65A, and (B) of FIG. 13 shows an internal pressure of the cuff 20 at this time. As shown in (A) of FIG. 13, the motor driver 28 applies, to the pump 60, the control voltage having a rectangular waveform with, for example, 10 seconds as one cycle.

In this case, the pump 60 is driven to output the pressurized air while the control voltage rises to a logic “1” level (for example, 12 V). Then, the pressurized air is supplied to the cuff 20 sequentially via the PP tube 64A connecting the pump 60 and the check valve 61, the PP tube 64B connecting the check valve 61 and the air buffer 62, and the PP tube 64D connecting the air buffer 62 and the cuff 20. As a result, the internal pressure of the cuff 20 increases and the cuff 20 inflates, applying an appropriate pressure to the finger 24 rolled with the cuff 20.

The pump 60 stops operating while the control voltage falls to a logic “0” level (for example, 0 V). As a result, internal pressures of the PP tube 64A connecting the pump 60 and the check valve 61, the PP tube 64B connecting the check valve 61 and the air buffer 62, and the PP tube 64D connecting the air buffer 62 and the cuff 20 decrease. Accordingly, the internal pressure of the cuff 20 also decreases and the cuff 20 contracts, reducing the pressure applied by the cuff 20 to the finger 24 rolled with the cuff 20.

In the blood collection device 3 according to the present embodiment, since the motor driver 28 of the control unit 23 applies the control voltage having the rectangular waveform to the pump 60, the cuff 20 repeats pressurization and depressurization for the finger 24 rolled with the cuff 20 in this manner, thereby providing the finger 24 rolled with the cuff 20 with the same effect as that when a person massages the finger 24. In the blood collection device 3 according to the present embodiment, an amount of bleeding from the finger 24 rolled with the cuff 20 can be increased by doing so, and more blood can be collected than when nothing is done.

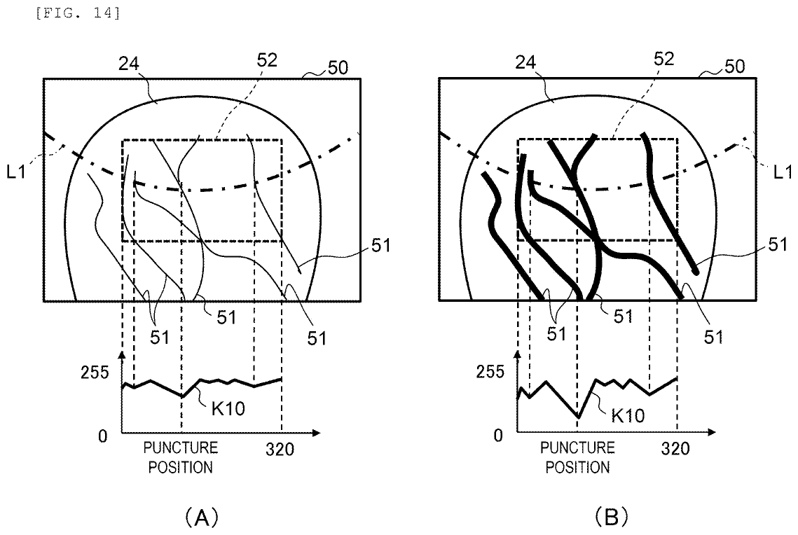

It is also possible to attain a vein enhancement effect in the fingertip image 50 by pressing the finger 24 of the subject with the cuff 20. That is, when the finger 24 of the subject is pressed, the finger vein 51 of the subject expands due to the pressure as shown in (B) of FIG. 14, and a contrast of a shadow of the finger vein 51 in the fingertip image 50 increases as compared with a case where the finger 24 is not pressed as shown in (A) of FIG. 14. As a result, when the puncture position candidate is extracted, a position where the puncture needle trajectory L1 intersects with the shadow of the finger vein 51 of the subject can be detected with higher accuracy.

(C) of FIG. 13 shows a voltage waveform of a control voltage applied by the control unit 23 to the infrared LED 26 via the GPIO interface 29 and the jumper wire 65B, and (D) of FIG. 13 shows brightness of the near-infrared light emitted from the infrared LED 26 at this time. As shown in (C) of FIG. 13, the control unit 23 applies, to the infrared LED 26, the control voltage having a rectangular waveform with, for example, 10 seconds as one cycle.

In this case, the infrared LED 26 emits near-infrared light having constant brightness according to a duty ratio of a period during which the control voltage rises to a logic “1” level (for example, 12 V) to a period during which the control voltage falls to a logic “0” level (for example, 0 V) under pulse width modulation (PWM) control. When the duty ratio is “1”, the brightness is the highest, and when the duty ratio is “0”, the light is turned off.

### (4) Effects of Embodiment

As described above, according to the blood collection system 1 in the present embodiment, the cuff 20 of the blood collection device 3 repeats pressurization and depressurization for the finger 24 rolled with the cuff 20, thereby providing the finger 24 rolled with the cuff 20 with the same effect as that when a person massages the finger 24, and increasing an amount of bleeding from the finger 24.

A contrast of a shadow of the finger vein 51 in the fingertip image 50 can be increased by applying a pressure to the finger 24 of the subject by the cuff 20 in this manner, and a position where the puncture needle trajectory L1 intersects with the shadow of the finger vein 51 of the subject can be detected with higher accuracy. As a result, the finger vein 51 of the subject can be accurately punctured with the puncture needle, and the amount of bleeding can be further increased.

Therefore, according to the blood collection system 1, it is possible to implement a blood collection system capable of collecting a sufficient amount of blood by improving blood collection efficiency.

In the blood collection system 1, since brightness adjustment, subject authentication, and determination of a puncture position for the puncture needle are executed using the finger vein 51 at the tip end portion beyond the first joint of the finger 24 of the subject, the device can be simplified and compact.

### (5) Other Embodiments

A case where the blood collection system 1 is configured with the subject authentication and blood collection control device 2 and the blood collection device 3 has been described in the above embodiment, but the invention is not limited to thereto, and all functions of the subject authentication and blood collection control device 2 may be provided in the blood collection device 3, and the blood collection system 1 may be configured only with the blood collection device.

A case where a function of authenticating the subject is provided in the blood collection system 1 has been described in the above embodiment, but the invention is not limited thereto, and only a blood collection function of collecting blood from the subject may be provided in the blood collection system 1.

Further, case where a control voltage having a rectangular waveform with 10 seconds as one cycle as shown in (A) of FIG. 13 is applied to the pump 60 of the cuff control unit 21 so as to change an internal pressure of the cuff 20 as shown in (B) of FIG. 13 has been described in the above embodiment, but the invention is not limited thereto, and a change pattern of the internal pressure of the cuff 20 may be a change pattern other than that shown in (B) of FIG. 13 as long as a pressure can be applied to the finger 24 of the subject so as to repeat pressurization and depressurization for the cuff 20 at a fixed cycle.

Further, a case where the finger 24 of the subject is irradiated with the near-infrared light has been described in the above embodiment, but the invention is not limited thereto, and light other than the near-infrared light may be used.

Further, a case where a plurality of (for example, three) puncture position candidates are extracted in step S35 of the puncture position determination processing described above with reference to FIG. 10 and one puncture position candidate is determined as the puncture position by the round robin method or the like according to luminance ranks of the plurality of puncture position candidates has been described in the above embodiment, but the invention is not limited thereto, and at least a puncture position candidate having a luminance rank different from the previous one may be determined as a current puncture position from among the plurality of puncture position candidates.

### INDUSTRIAL APPLICABILITY

The invention can be applied to a blood collection system used for collecting blood of a subject.

### REFERENCE SIGNS LIST

1: blood collection system
2: subject authentication and blood collection control device
3: blood collection device
13: CPU
16: subject-oriented application
17: blood collection application
18: authentication database
20: cuff
21: cuff control unit
22: blood collection unit
23: control unit
24: finger
25: puncturing and blood collection mechanism
26: infrared LED
27: infrared night vision camera
30: rotary table
30A: holder
32: finger rest
32C: opening
50: fingertip image
51: finger vein
52: processing region
L1: puncture needle trajectory
L2: trajectory marker
60: pump

1. A blood collection system used for collecting blood of a subject, the blood collection system comprising:
a puncturing and blood collection mechanism configured to puncture a finger of the subject with a puncture needle;
a cuff configured to be attached to the finger of the subject in a rolling manner; and
a cuff control unit configured to supply pressurized air to the cuff so as to inflate the cuff and apply a predetermined pressure to the finger of the subject.

2. The blood collection system according to claim 1, wherein
the cuff control unit supplies the pressurized air to the cuff in a predetermined pattern such that an internal pressure of the cuff repeatedly increases and decreases.

3. The blood collection system according to claim 1, further comprising:
a light source configured to irradiate the finger of the subject with light;
a camera configured to image the light that passes through the finger of the subject; and
a blood collection control unit configured to determine a puncture position for the puncture needle by the puncturing and blood collection mechanism based on a captured video from the camera, wherein
the blood collection control unit determines the puncture position so as to puncture a finger vein of the subject with the puncture needle based on the captured video from the camera.

4. The blood collection system according to claim 3, wherein
the blood collection control unit determines, as a current puncture position, at least a position different from a previous position on the finger of the subject.

5. The blood collection system according to claim 3, wherein
the light source irradiates the finger of the subject with near-infrared light as the light, and
the camera is an infrared night vision camera.

6. The blood collection system according to claim 3, wherein
the blood collection control unit authenticates the subject using the finger vein of the subject imaged by the camera.

7. The blood collection system according to claim 6, wherein
the blood collection control unit authenticates the subject and determines the puncture position using the finger vein on a fingertip end side with respect to a first joint of the finger of the subject.

8. A blood collection method executed in a blood collection system used for collecting blood of a subject, the blood collection method comprising:
a first step in which the blood collection system punctures a finger of the subject with a puncture needle; and
a second step in which the blood collection system supplies pressurized air to a cuff attached to the finger of the subject in a rolling manner so as to inflate the cuff and apply a predetermined pressure to the finger of the subject.

9. The blood collection method according to claim 8, wherein
in the second step, the pressurized air is supplied to the cuff in a predetermined pattern such that an internal pressure of the cuff repeatedly increases and decreases.

10. The blood collection method according to claim 8, wherein
the blood collection system includes
a light source configured to irradiate the finger of the subject with light, and
a camera configured to image the light that passes through the finger of the subject, and
in the first step, the blood collection system
determines the puncture position so as to puncture a finger vein of the subject with the puncture needle based on a captured video from the camera, and
punctures the determined puncture position with the puncture needle.

11. The blood collection method according to claim 10, wherein
in the first step, the blood collection system determines, as a current puncture position, at least a position different from a previous position on the finger of the subject.

12. The blood collection method according to claim 10, wherein
the light source irradiates the finger of the subject with near-infrared light as the light, and
the camera is an infrared night vision camera.

13. The blood collection method according to claim 10, wherein
in the first step, the blood collection system authenticates the subject using the finger vein of the subject imaged by the camera.

14. The blood collection method according to claim 13, wherein,
in the first step, the blood collection system authenticates the subject and determines the puncture position using the finger vein on a fingertip end side with respect to a first joint of the finger of the subject.
